(19) United States
(12) Patent Application Publication
(10) Pub. No.: US 2013/0109975 A1
(43) Pub. Date: May 2, 2013

(54) MONOLITHIC DEVICE

(71) Applicants: Mario Ettore Giardini, St Andrews (GB); Thomas Fraser Krauss, St Andrews (GB); Andrea Di Falco, St Andrews (GB)

(72) Inventors: Mario Ettore Giardini, St Andrews (GB); Thomas Fraser Krauss, St Andrews (GB); Andrea Di Falco, St Andrews (GB)

(73) Assignee: UNIVERSITY COURT OF THE UNIVERSITY OF ST ANDREWS, St Andrews (GB)

(21) Appl. No.: 13/639,433

(22) PCT Filed: Apr. 6, 2011
(86) PCT No.: PCT/GB2011/000528

§ 371 Date: Jan. 4, 2013

(30) Foreign Application Priority Data
Apr. 9, 2010 (GB) 1005930.1

(51) Int. Cl. A61B 5/00

(52) U.S. Cl. CPC A61B 5/0075
USPC 600/473; 600/476

(57) ABSTRACT

A monolithic device for the non-invasive measurement of optical parameters relating to a biological tissue comprising: at least one light emitter that emits light having a range of wavelengths and/or at least one detector for detecting light in a range of wavelengths transmitted or backscattered by the tissue, wherein one or more structures with sub-wavelength features is formed on the at least one emitter and/or the at least one detector.

### FIELD OF THE INVENTION

The present invention relates to a system and method for monitoring light transmission and/or backscattering. In particular, the present invention relates to a surgical or medical device for sampling tissue using light.

### BACKGROUND OF THE INVENTION

Light transmission and/or backscattering, typically in the near-infrared, is a well known technique for monitoring blood and other biological tissue constituents. It allows, for example, the degree of oxygenation of such tissues to be established. This is because haemoglobin and myoglobin have different near-infrared optical absorption spectrum depending on whether they are in an oxygenated or deoxygenated state. The oxygenation state can be determined by shining light on the tissue and observing the transmitted or backscattered light intensity. As another example, the content of cytochrome aa₃ oxydase in tissue can be determined in a similar way.

Monitoring oxygenation levels is very useful, for example during surgery, as tissue needs to be interrogated in order to establish whether it is correctly perfused by blood. Other applications include emergency care medicine, for the determination of the oxygenation state of brain tissue; sports medicine and rehabilitative cardiology, for the determination of the oxygenation state of muscle haemodynamics and of capillary contractility; vascular surgery, for the determination of blood vessel elasticity by observation of the response of vascularised tissue to adequate stimuli; catheterised tools, as a navigation aid via the identification of different types of tissues through their optical backscattering and/or transmission properties.

U.S. Pat. No. 5,807,261 describes a tool for non destructive interrogation of tissue. This has a light source and light detector mounted directly on the tool or mounted remotely and guided to the surgical field using fibre optic cables. Various source and detector configurations are described.

### SUMMARY OF THE INVENTION

According to a first aspect of the present invention, there is provided a device for non-invasive measurement of biological parameters that has an emitter that emits radiation that has a range of wavelengths, wherein features are provided on the emitter, the features having at least one dimension smaller than that of the wavelengths emitted by the emitter, so that light output from the device is determined by the sub-wavelength features. This allows the spectral response of the device to be defined by geometry alone.

The sub-wavelength features on the emitter may have one or more dimensions that are less than or equal to half the central wavelength, i.e. λ/2, of the wavelengths that can be emitted. Typically, the subwavelength features have one or more dimensions in the range 10 nm to 350 nm.

The device may have a detector that detects radiation over a range of wavelengths, wherein features having at least one dimension smaller than the wavelengths that can be detected are provided on the detector, so that the wavelength detected is determined by the sub wavelength features. This allows the spectral response of the device to be defined by geometry alone.

The sub-wavelength features on the detector may have one or more dimensions that are less than or equal to half the central wavelength, i.e. λ/2, of the wavelengths that can be detected. Typically, the subwavelength features have one or more dimensions in the range 10 nm to 350 nm. The sub wavelength features may form part of one or more gratings.

The device of the invention has at least one light emitter and/or at least one detector for detecting light transmitted or backscattered by the tissue, wherein one or more structures with sub-wavelength features is formed on the at least one emitter and/or the at least one detector. The sub wavelength features may form part of one or more gratings.

At least two emitters may be provided. The at least two emitters may be provided on a single substrate, thereby forming a monolithic device. The sub wavelength features on the at least two emitters may be such that the light emitted by them is of different wavelengths. The sub wavelength features may form part of one or more gratings.

At least two detectors may be provided. The at least two detectors may be provided on a single substrate, thereby forming a monolithic device.

At least one emitter and at least one detector may be provided. The at least one emitter and the at least one detector may be provided on a single substrate, thereby forming a monolithic device.

The at least one emitter and the at least one detector may be made of different material. The at least one emitter may be provided on a substrate of a first material and the at least one detector is provided on a substrate of a second material.

The at least one emitter and/or at least one detector may comprise semiconductor material. The semiconductor material may be inorganic.

The at least one emitter and/or at least one detector may comprise emitting or absorbing dyes.

Light from each emitter may be in the infrared region. Light from each emitter may have a bandwidth in the wavelength range of 10-140 nm, preferably 20-100 nm.

The emitter may comprise a light emitting diode.

The device may be implantable in the human or animal body. The device may be coated in a non-degradable bio-compatible material that is transparent at the emitted wavelength. The device may include a transmitter for transmitting signals from the implantable device to a remote receiver.

According to another aspect of the invention, there is provided a surgical or medical device that includes a device according to the first aspect. The surgical/medical device may be an endoscope or a laproscope.

### BRIEF DESCRIPTION OF THE DRAWINGS

Various aspects of the invention will now be described by way of example only and with reference to the accompanying drawings, of which:

FIG. 1(a) is a cross section through a monolithically formed backscattering/transmission device;

FIG. 1(b) is a cross section through a subwavelength grating part of the device of FIG. 1(a);

FIG. 2 shows spectra of two sources used in the device of FIG. 1;

FIG. 3 is a schematic diagram of a backscattering measurement tool for measuring optical characteristics of tissue, and

FIG. 4 is schematic diagram of a transmission measurement tool for measuring optical characteristics of tissue.

### DETAILED DESCRIPTION OF THE DRAWINGS

In order to simplify the construction of backscattering and transmission measurement tools, the sources and/or the detectors are assembled directly on the sensing element, and on a single common substrate. This allows single devices of the order of a few mm² or smaller to be made incorporating multiple sources and/or detectors. This device requires no further assembly of optical components and is easier to integrate into a surgical instrument than systems composed of separate parts, such as individual sources, detectors and optical fibres.

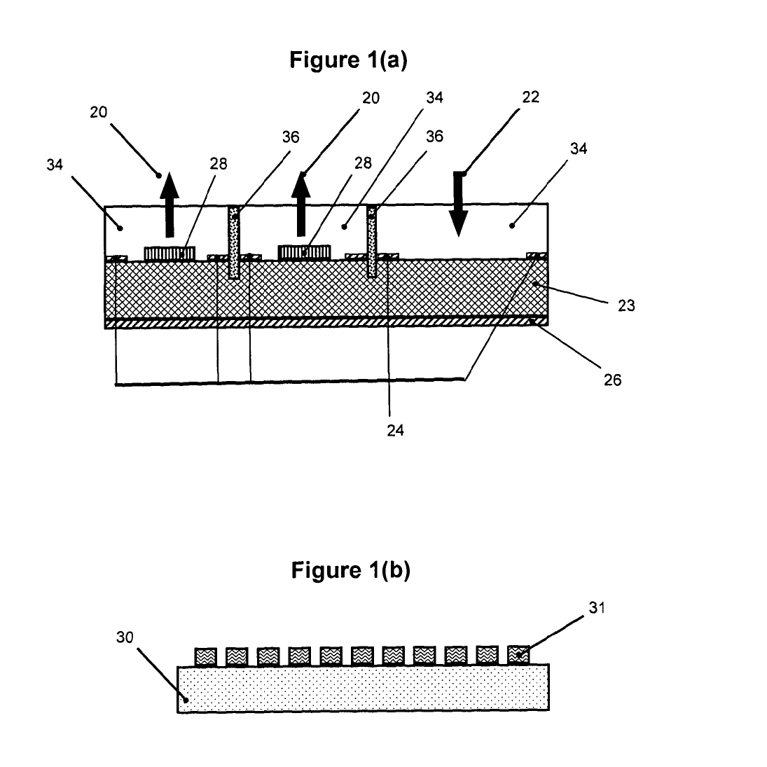

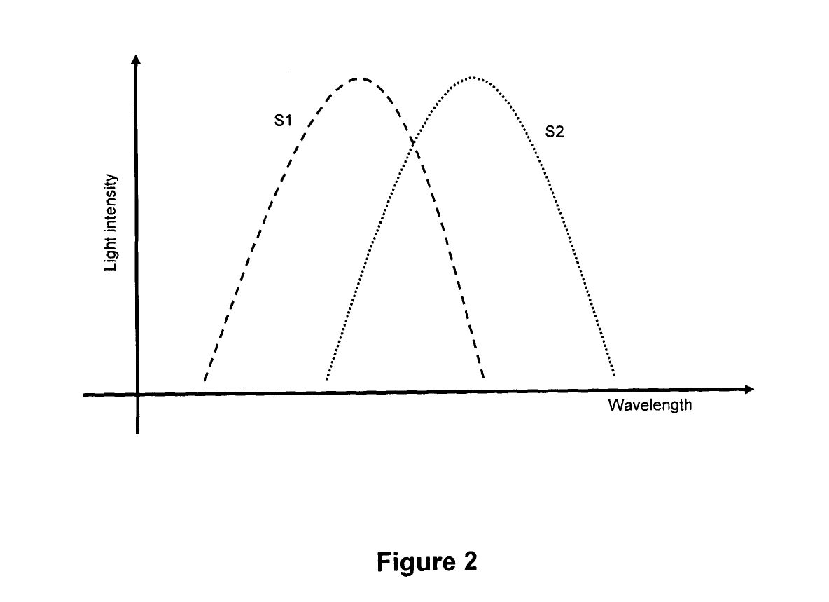

FIGS. 1(a) and 1(b) show an example of a monolithically integrated device for use in an optical measurement tool. This has two emitters 20 and a single detector 22 fabricated on the same semiconductor substrate. The semiconductor substrate is chosen to operate preferentially around 780 nm wavelength emission and absorption wavelength. As an example, the substrate is made of GaAs and/or composites of GaAs. The emitter comprises a light emitting structure, for example an LED or a resonant cavity LED with a relatively broad emission range, i.e. having a wavelength bandwidth in the range range 10-140 nm, preferably in the range 20-100 nm. FIG. 2 shows examples of broadband spectra for light emitted from such emitters.

As an alternative to forming the light emitting and/or light detecting structures using semiconductor material on a semiconductor substrate, the emitters and/or detectors may instead be formed by depositing different active and detecting materials on a common substrate. For example, the active and detecting materials may include emitting or absorbing dyes, and/or semiconductor Nanocolloids, like CdS or CdSe.

The areas that are operated as emitters are separated electrically, so they can be driven as electrically independent units using separate contacts, for example top contacts 24 and bottom contact 26. The detector 22 is also electrically driven independently through separate contacts, i.e. top contact 24 and bottom contact 26. The contacts can be formed in any suitable way, for example by plasma evaporation of two or more layers of metal chosen between Ni, Ge, Au, Cr, each with thicknesses between 10 and 300 nm, depending on the substrate properties. The bottom contact 26 may be a shared or common contact.

Each emitter and/or detector is covered by a subwavelength grating 28 in order to modify the emission/detection spectral response. The sub wavelength grating has a periodic structure, for example a series of lines or ridges. Each feature or ridge of the sub-wavelength grating may have one or more dimensions, usually a width, that is less than or equal to half the central wavelength, i.e. λ/2, of the wavelengths that can be emitted or detected by the associated emitter or detector. Typically, the subwavelength features have one or more dimensions in the range 10 nm to 350 nm. The subwavelength gratings are an integral part of the device and determine the wavelength selectivity solely by a geometrical property of the device exhibiting features on the subwavelength size scale. These may be created, for example, by a lithographically created pattern. A typical example is shown in FIG. 1(b).

To form the subwavelength structures, firstly a low refractive index buffer 30 is deposited, with thickness between 0 and 100 μm, the range 100 nm to 500 nm being preferred. The buffer material should not be absorbing at the emission wavelength and its thickness is controlled with nanometric precision (+−10 nm). The buffer material, if polymeric, can be applied, for example, by dissolving it in a solvent, by spinning the solution onto the emitters and/or the detectors, and by evaporating the solvent. Preferred polymers are PMMA, SU8 or Polymide. Other suitable materials, for example, SiO₂ or amorphous silicon, could be deposited on the emitters and/or detectors using for example thermal or plasma evaporation or sputtering.

On top of the buffer is deposited a transparent layer that has a higher refractive index 31 than the buffer, see FIG. 1(b). For example, the transparent layer could be Si₃N₄ or amorphous silicon, or a high index polymer could be deposited using for example spinning, evaporation and sputtering. The thickness of this layer is typically below 1 μm. This layer is patterned to define the sub-wavelength features, for example a grating, as shown in FIG. 1(b). The patterned area could be as small as few μm² to as large as covering the whole emission or detection surface. Each of these subwavelength structures alters the wavelength range emitted by the sources and/or detected by the detectors, such that each device acts as a spectrally separate emitter. A typical emission bandwidth that can be achieved with this is method is 10-20 nm.

Different areas of the substrate can be patterned in different ways. Certain areas could be patterned to serve as detectors, others to serve as emitters. The emission area could be shaped in any geometrical shape, with typical surface with dimension between 10 μm² to several mm². The total detecting area typically covers a surface in the range from a few 10 μm² of several mm².

The device could be coated using a suitably chosen biocompatible material 34 (such as, for example, biocompatible silicone, cyanoacrylate or epoxy resins), as shown in FIG. 1(a). This is transparent at the relevant wavelengths. Typical, thicknesses are in the range of 10 nm to 1 mm.

Optical separation between the single emitters and the detectors is achieved via cuts 36 in the coating material 34 which may be as deep as to reach the substrate and realized together with the electrical separation voids. The cuts, which could be as wide as few um up to several mm, could be left empty or backfilled with suitable material.

In use, the different emitters can be modulated with different frequencies or modulation codes. The different wavelength signals can be identified by the detection circuit and the received data processed accordingly. Any suitable modulation technique can be used.

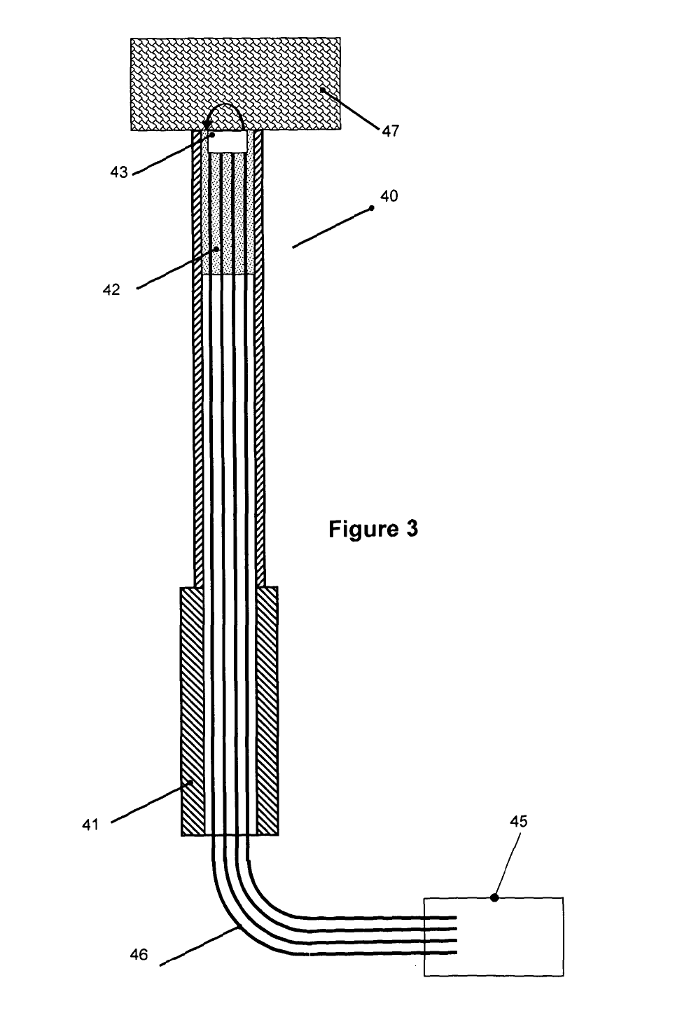

FIG. 3 shows a backscattering measurement tool, such as a laparoscopic tool 40, for interrogating tissue for oxygenated and deoxygenated haemoglobin content using a monolithic source/detector. This has a hollow metallic shaft (typical length 40 cm) with a handle 41 and a tip 42. A monolithic device 43, including sources and detectors, as described previously, is located at the tip 42 of the tool 40. It can be secured to the tip 42 in any suitable way, for example using biocompatible glue. Control electronics 45 are connected through the handle 41 to the monolithic device 43. Electrical cables 46 to and from the electronics 45 drive the source(s) and read the backscattered light collected by the detector(s). In use, the tool 40 is positioned so that it touches the tissue to be investigated 47 with the distal tip 42. The electronics 45 identifies how much light coming from each source is backscattered, and applies the algorithms necessary to extract the relevant data.

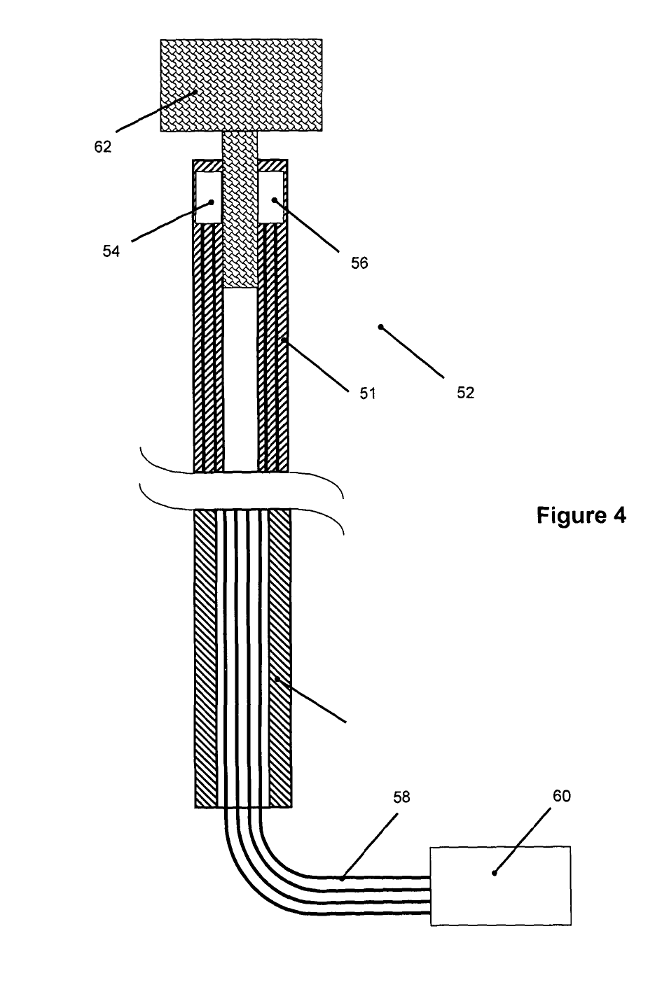

FIG. 4 shows a transmission measurement tool. This has sources 54 and detectors 56 coupled to the grasping tool 51 at the tip of a surgical gripper 52. The sources 54 are provided on single substrate, so that they form a single monolithic device. At least one of the sources 54 has sub-wavelength features formed on it. The detectors 56 are provided separately on another single substrate, so that they too form a single monolithic device. At least one of the detectors 56 has sub-wavelength features formed on it. In this case, the substrate used for the sources 54 and the substrate used for the detectors may be made of the same or different material.

The sources 54 and detectors 56 are positioned on opposite sides of the grasping tool 51, but facing each other, so that light from the sources 54 is directed towards the detectors 56. Electrical cables 58 connect the sources 54 and detectors 56 to an electronic unit 60, which drives the sources 54 and collect the signals from the detectors 56. In use, tissue 62 is grasped between the faces of the grasping tool 51 and light is emitted from the sources 54, passes through the tissue 62 and into the detectors 56 opposite.

The monolithic device of the present invention is compact, robust and simple. It can be readily incorporated into medical or surgical devices such as endoscopes, laproscopes and implantable devices. It can be used in any optical spectroscopy technique that can benefit from the application of multiple sources to biological tissue, and from the assignment, on one or more detectors, of the signal contribution deriving from each source. For example, the invention could be applied to transmission and/or backscattering spectroscopy, fluorescence spectroscopy, Raman scattering.

A skilled person will appreciate that variations of the disclosed arrangements are possible without departing from the invention. For example, although FIGS. 3 and 4 show the monolithic devices of the invention as part of surgical tools, the devices could be designed to be implantable in the human or animal body. In this case, the device would be coated with a non-degradable bio-compatible material that is transparent at the emitted wavelength. The device would also include a power source for powering the components, and optionally a transmitter for transmitting signals to a remote receiver. Accordingly, the above description of the specific embodiment is made by way of example only and not for the purposes of limitation. It will be clear to the skilled person that minor modifications may be made without significant changes to the operation described.

The work leading to this invention has received funding from the Commission of the European Communities Information Society and Media Directorate-General Information and Communication Technologies—Seventh Framework Programme, a Collaborative Project entitled “Array of Robots Augmenting the KiNematics of Endoluminal Surgery” (ARAKNES) ([FP7/2007-2013) under grant agreement no 224565.

1. A monolithic device for non-invasive measurement of optical parameters relating to a biological tissue, the monolithic device comprising at least one of:
at least one light emitter that emits light having a range of wavelengths or
at least one detector for detecting light in a range of wavelengths transmitted or backscattered by the tissue, wherein one or more structures with sub-wavelength features is formed on the at least one light emitter or the at least one detector.

2. A device as claimed in claim 1 wherein at least two light emitters are provided on the monolithic device.

3. A device as claimed in claim 2 wherein the sub-wavelength features on the at least two emitters are such that light emitted by the at least two emitters is of different wavelengths.

4. A device as claimed in claim 1 wherein at least two detectors are provided on the monolithic device.

5. A device as claimed in claim 1 wherein the at least one light emitter and the at least one detector are provided on the monolithic device.

6. A device as claimed in claim 5 wherein the at least one light emitter and the at least one detector are made of different material.

7. A device as claimed in claim 1 wherein the at least one light emitter or the at least one detector comprise semiconductor material.

8. A device as claimed in claim 7 wherein the semiconductor material is inorganic.

9. A device as claimed in claim 1 wherein the at least one light emitter or the at least one detector comprise emitting or absorbing dyes.

10. A device as claimed in claim 1 wherein the sub-wavelength features form part of one or more gratings.

11. A device as claimed in claim 1 wherein light from each of the at least one light emitter is in an the infrared region.

12. A device as claimed in claim 1 wherein light from each of the at least one light emitter has a bandwidth in a the wavelength range of 10-140 nm, preferably 20-100 nm.

13. A device as claimed in claim 1 wherein the at least one light emitter comprises a light emitting diode.

14. A device as claimed in claim 1 that is implantable in a the human or an animal body.

15. A device as claimed in claim 14, wherein the monolithic device is coated in a non-degradable bio-compatible material that is transparent at an emitted wavelength.

16. A device as claimed in claim 14 comprising a transmitter for transmitting signals from the implantable device to a remote receiver.

17. A device as claimed in claim 1 wherein the sub-wavelength features have one or more dimensions in the range 10 nm to 350 nm.

18. A medical device, wherein the medical device includes at least one of:
at least one light emitter that emits light having a range of wavelengths; or
at least one detector for detecting light in a range of wavelengths transmitted or backscattered by the tissue, wherein one or more structures with sub-wavelength features is formed on the at least one light emitter or the at least one detector,
wherein the medical device is implantable in a human or an animal body.

19. A medical device as claimed in claim 18 wherein the device is an endoscope or a laproscope.
